(19) United States
(12) Patent Application Publication
(10) Pub. No.: US 2015/0343240 A1
(43) Pub. Date: Dec. 3, 2015

(54) TEST OBJECT FOR THE CORRECTION OF MOTION ARTEFACTS OF A PORTAL IMAGER EQUIPPING AN EXTERNAL RADIATION THERAPY TREATMENT DEVICE WHEN THE EXTERNAL RADIATION THERAPY TREATMENT DEVICE IS MOVING

(71) Applicant: QUALIFORMED, La Roche-sur-Yon (FR)

(72) Inventor: Stephane BEAUMONT, Nieul Le Dolent (FR)

(21) Appl. No.: 14/729,522

(22) Filed: Jun. 3, 2015

(30) Foreign Application Priority Data
Jun. 3, 2014 (FR) 1455040

(51) Int. Cl. A61N 5/10

(52) U.S. Cl. CPC A61N 5/1075; A61N 2005/1076

(57) ABSTRACT

Disclosed is a test object for an external radiation therapy treatment device, the treatment device including a rotating gantry carrying at one end a radiation head and, at the other end, a portal imager, the test object including a radiologically translucent plate within which at least one radiopaque member is embedded, the at least one radiopaque member forming a geometrical pattern having at least two points and the center of the geometrical pattern corresponding to the center of the radiologically translucent plate, and a fastening element to attach the test object on the radiation head.

### CROSS REFERENCE TO RELATED APPLICATIONS

Applicant claims priority under 35 U.S.C. 119 of French patent application no. 1455040 filed on Jun. 3, 2014.

### BACKGROUND OF THE INVENTION

The present invention relates to the field of external radiation therapy treatment, and particularly relates to test objects and methods for correcting measures performed on the images corresponding to the performance tests of an external radiation therapy treatment device to take into account motion artefacts, with respect to the radiation source of the treatment device, of a portal imager equipping said external radiation therapy treatment device when the gantry thereof is moving.

In an external radiation therapy treatment, tumors are sterilized by irradiating them at high dose using multiple ionizing radiation beams (most often, x-rays, but other radiations such as electron, neutron or ion beams are also used), which are concentric or not. The positioning of the patient with respect to the radiation beams is essential: indeed, an incorrect positioning of the patient with respect to the radiation beams will result on the one hand in an incomplete irradiation of the tumor, which can induce a disease recurrence, and on the other hand in an irradiation of healthy tissues close to the tumor, which can cause serious complications for the patient.

To ensure a targeting of the radiation beams on the tumor, several quality control tests of the external radiation therapy treatment devices were designed and are performed on the external radiation therapy treatment device before the external radiation therapy treatment of a patient.

A conventional external radiation therapy treatment device, shown for example in FIG. 4, comprises a rotating gantry carrying at one end a radiation head comprising a radiation source, the radiation head ending by a collimator which delimits the radiation beam, and at the other end an output imager called “portal imager” which can make digital radiographs of an object arranged between the collimator and the portal imager, generally on a treatment table, also called “patient support”. The portal imager is thus used either to control the position of the patient before his/her external radiation therapy treatment, or to perform performance tests of the external radiation therapy treatment device.

Such performance tests of the external radiation therapy treatment device are, for example, described in the “Quality Assurance of Medical accelerators” report of the task group 142 of the American Association of Physicists In Medicine (AAPM) (TG 142 of AAPM).

The radiation head of the external radiation therapy treatment device projects, toward the patient support and the portal imager, a radiation beam for the treatment of the patient, the radiation beam passing through the collimator which is the end part of the radiation head and allows to delimit the radiation beam according to many configurations.

When the gantry of the external radiation therapy treatment device is rotating, the head of the device which contains the radiation source and the collimator being cantilevered, the high weight of the head causes the radiation source to longitudinally move forward (in the case where the head is at the top) or backward (in the case where the head is at the bottom) with respect to a vertical plane perpendicular to the rotation axis of the gantry of the external radiation therapy treatment device. The portal imager arranged on the gantry opposite to the radiation source is subjected to mechanical stresses of the same type, with very different intensities due to the fact that the portal imager is much more lightweight than the head of the external radiation therapy treatment device, the longitudinal and vertical motion artefacts of the portal imager with respect to the axis of the radiation beam being mainly caused by the flexion of the gantry under the conjugated weight of the radiation head and of the portal imager. The vertical motion artefact is a consequence of the fact that the radiation source is not at a constant distance from the portal imager when the gantry rotates. The longitudinal and transverse motion artefacts are a consequence of the fact that the projection on the portal imager of axis of the radiation beam is not strictly at the same spot when the gantry rotates.

In addition, when the gantry rotates, the portal imager, which can generally move in the three spatial dimensions by means of engines, gears and endless screws, has mechanical clearances, the strongest influence of which can be found on transverse motion artefacts of the portal imager with respect to the axis of the radiation beam.

Thus, when the gantry rotates, the distance between the radiation source and the portal imager is not constant, and the longitudinal and transverse alignments of the portal imager with respect to the radiation source vary, the typical intensity values of these motion artefacts being, for a high-efficiency treatment device, of about 1 mm in the longitudinal direction, 1 mm in the transverse direction and 2 mm in the vertical direction.

It is thus necessary to correct the motion artefacts of the portal imager when it is moving with the rotation of the gantry of the external radiation therapy treatment device, as part of performance tests of the multileaf collimator (MLC) used for both delimiting the radiation beam and modulating its intensity during the treatment.

The French patent application FR2956979 discloses a test object and a method using such test object allowing to control the coincidence of light fields and radiated fields on an external radiation therapy treatment device, the test object being constituted by a body with an electronic density and at least one member integral with a body surface or embedded therein and able to be distinguished from the body by ionizing radiation beam imaging. However, this test object does not allow to accurately measure the motion artefacts of the portal imager of the external radiation therapy treatment device when it is moving with the rotation of the gantry, so as to correct said motion artefacts of the portal imager.

### BRIEF SUMMARY OF THE INVENTION

The present invention is intended to solve the disadvantages of the prior art by providing a test object to accurately measure the motion artefacts of the portal imager of an external radiation therapy treatment device when the device is moving with the rotation of the gantry of the external radiation therapy treatment device, so as to correct said motion artefacts of the portal imager with respect to the rotation angle of the gantry, said test object comprising a radiologically translucent plate (also referred to as radiotranslucent plate) in which several radiopaque members are embedded within the thickness of the radiologically translucent plate, the test object being attached at the output of the radiation head of the external radiation therapy treatment device and as close as possible thereto, the test object also having a simple structure, and thereby being easy to produce and low cost.

The present invention also relates to two methods for measuring and correcting measures performed in the images corresponding to the performance tests of an external radiation therapy treatment device to take into account motion artefacts of a portal imager equipping an external radiation therapy treatment device when the device is in movement, both methods using the test object according to the present invention, the first method requiring a pre-test for measuring the corrections to be performed during the performance test of the external radiation therapy treatment device, and the second method performing the measures of the corrections to be performed directly during the performance test of the external radiation therapy treatment device.

The present invention thus relates to a test object for an external radiation therapy treatment device, said external radiation therapy treatment device comprising a rotating gantry carrying at one end a radiation head and comprising a radiation source and, at the other end, a portal imager, said test object being intended to correct motion artefacts of the portal imager with respect to the radiation source of the external radiation therapy device during a movement of the external radiation therapy treatment device, wherein said test object comprises a radiologically translucent plate within the thickness of which at least one radiopaque member is embedded, said radiopaque member(s) forming a geometrical pattern having at least two points and the center of said geometrical pattern corresponding to the center of said radiologically translucent plate, and fastening means allowing to fasten the test object to the radiation head.

Thus, the test object has a simple structure and can therefore be produced at a low cost.

The radiopaque member(s) can be a wire, a ball or any member with a small size able to be embedded within the thickness of the radiologically translucent plate. The expression “radiologically translucent” means that the plate is radiologically translucent or close to radiologically translucent, most of the radiation passing through the plate. The radiologically translucent plate thereby allows the high energy radiation produced by the external radiation therapy treatment device to pass therethrough, the maximum attenuation of the radiation passing through the plate being of 10%.

The geometrical pattern is formed in the plane of the radiologically translucent plate and can be a segment of two points, a triangle, a square, a rectangle, a regular polygon or any geometrical pattern whose center can be easily determined geometrically. It can be noted that the geometrical pattern can be continuous (a closed loop wire embedded within the thickness of the radiologically translucent plate and forming said geometrical pattern) or discrete (balls arranged at the vertexes of the geometrical pattern).

The at least one radiopaque member allows to precisely determine the position of their image on the portal imager after irradiating the test object by a radiation source.

The radiopaque member(s) can preferably be arranged on at least two of the edges of the radiologically translucent plate so as not to block the center part of the image which is useful for the performance test of the external radiation therapy treatment device.

Preferably, all radiopaque members are embedded within the thickness of the radiologically translucent plate at the same depth with respect to the face of the radiologically translucent plate facing the radiation head, all said radiopaque members being thus disposed at the same distance from the radiation source of the radiation head.

The fastening means allow to attach the test object to the radiation head and thus to make the test object integral with the radiation head, thereby allowing to perfectly characterize the relative changes of position between the radiation head (and therefore the radiation source) and the portal imager during a movement of the external radiation therapy treatment device.

Advantageously, the test object is immediately attached at the output of the radiation head for integrally moving therewith.

The fastening means can be constituted by a hollow metal frame on which the radiologically translucent plate is attached, said frame being adapted to be inserted in a pair of rails arranged at the output of the radiation head of the external radiation therapy treatment device, the test object being thus as close as possible to the radiation source of the radiation head.

However, without departing from the scope of the present invention, the fastening means can be screws which are screwed in the radiation head, an adhesive, or more generally any means allowing to make, preferably releasably, the test object integral with the radiation head.

According to a first embodiment of the invention, the radiologically translucent plate carries at least two radiopaque balls.

Thus, the test object according to the first embodiment has a simple structure and is easy to produce.

In addition, many geometries can be adapted for positioning the balls in the radiologically translucent plate, such as a line formed by two balls, an equilateral triangle formed by three balls, or a square or rectangle.

According to a second embodiment of the invention, the radiologically translucent plate carries two parallel rows of balls, each row of balls comprising at least two balls, both rows of balls comprising the same number of balls spaced by the same spacing, such that the line connecting two balls facing each other in different rows is perpendicular to the direction of the rows of balls.

A “ladder” pattern is thus formed, each row constituting one of the posts of the ladder, and each ball representing one of the ends of a rung of the ladder.

Thus, even if the test object according to the second embodiment is more difficult and therefore expensive to produce than the test object according to the first embodiment, this test object according to the second embodiment still has a relatively simple structure.

Both parallel rows of balls are adapted for an external radiation therapy treatment device comprising a collimator having two leaf banks allowing to produce a longitudinal sliding slit with respect to the rotation angle of the gantry, only one pair of balls of both parallel rows of balls being visible by the radiation source for a given rotation angle of the gantry.

According to a third embodiment of the invention, the test object further comprises at least four radiopaque balls forming a square or rectangle, the contour of which surrounds both rows of balls, the center of the square or rectangle formed by the four radiopaque balls corresponding to the barycenter of both rows of balls, namely the intersection of the diagonals of the rectangle whose vertexes are the four end balls of both rows of balls.

The test object according to the third embodiment is a combination of the test objects of the first and second embodiments.

According to a particular feature of the invention, the radiologically translucent plate is made of radiologically translucent plastic material, conferring a radiologically translucent property to the plate to allow high energy radiation to pass through the plate and also ensuring a dimensional stability to the plate under the repeated effect of radiation. The radiologically translucent plastic material is preferably polycarbonate.

Thus, the radiologically translucent plate has a low cost, is easy to perforate for the insertion of the radiopaque members and, contrary to the radiopaque members, is radiologically translucent and allows the passage therethrough of ionizing rays emitted from the radiation source of the radiation head of the external radiation therapy treatment device.

According to a particular feature of the invention, the thickness of the radiologically translucent plate is constant and comprised between 3 and 10 mm, preferably 6 mm.

Thus, the thickness of the radiologically translucent plate being constant, all ionizing rays emitted by the radiation source cross substantially the same thickness of radiologically translucent plate.

In addition, the thickness range from 3 to 10 mm allows the radiologically translucent plate to be sufficiently rigid not to be deformed when the gantry rotates, and not to be too thick so as to maintain a good contrast on the images for the radiopaque balls comprised therein.

According to a particular feature of the invention, the radiopaque members are made of tungsten carbide, but can also be made of any other metal or material with an electronic density significantly higher, namely at least two times higher, than the electronic density of the radiologically translucent plate.

Thus, the radiopaque members have good radio-opacity properties and can be easily spotted on the image formed, thereby allowing a computer processing of the image formed.

It can be noted that the ball shape is the preferred shape for the radiopaque members, as the spherical shape of the surface ensures that the image by the radiation beam of the radiopaque members detected by the portal imager is the same, regardless of the divergence conditions of the radiation beam and of the size of the radiation beam.

According to a particular feature of the invention, each radiopaque ball is embedded in the radiologically translucent plate such that the center of each ball is at a distance from the face of the radiologically translucent plate substantially equal to the radius of the radiopaque ball.

Thus, all balls are at the same depth with respect to the face of the radiologically translucent plate facing the radiation head, and thus all are at the same distance from the radiation source of the radiation head.

The diameter of the balls is large enough such that the images of the balls after the radiation can be viewed by the portal imager of the external radiation therapy treatment device. Especially, the diameter of the balls can be between 1 and 10 mm.

According to the first embodiment, the diameter of the balls is between 2 and 6 mm and, advantageously, can be of 4 mm.

According to the second embodiment, the diameter of the balls is between 1 and 5 mm and, advantageously, can be of 2 mm.

According to the third embodiment, the diameter of the four balls forming a square or rectangle surrounding both rows of balls is between 2 and 6 mm and, advantageously, can be of 4 mm.

Thus, all balls of both rows of balls are at the same depth with respect to the upper surface of the radiologically translucent plate facing the radiation head, and are thus at the same distance from the radiation source of the radiation head.

In addition, the diameter of the balls of both rows of balls is large enough such that the images of the balls after the radiation can be viewed by the portal imager of the external radiation therapy treatment device, the diameter of the balls also being small enough such that a ball can be contained in the radiation slit of the collimator of the external radiation therapy treatment device.

According to a particular feature of the invention, a reticule is engraved at the face of the radiologically translucent plate intended to face the radiation head, said reticule representing the center of the radiologically translucent plate.

Thus, the reticule engraved on the radiologically translucent plate allows to visually identify the center of the radiologically translucent plate, which is useful during the installation of the test object on the external radiation therapy treatment device by a user.

Advantageously, each radiopaque member of a test object according to the present invention is at a minimum distance from the center of the radiologically translucent plate comprised between 50 mm and 70 mm, preferably 62 mm.

Thus, it is ensured that no radiopaque member will appear in the useful (center) part of the image on the portal imager, while ensuring that each radiopaque member is in the radiation beam, so as to be visible on the edges of the image formed on the portal imager, for performing measures on the geometrical pattern formed in the image of the portal imager by the radiopaque member(s).

The invention also relates to a method with a pre-test for measuring and correcting the measures performed in the images corresponding to the performance tests of an external radiation therapy treatment device to take into account the motion artefacts, with respect to the radiation source of the external radiation therapy treatment device, of a portal imager equipping the external radiation therapy treatment device when the latter is moving, the external radiation therapy treatment device further comprising a rotating gantry carrying at one end a radiation head comprising a radiation source and, at the other end, a portal imager, wherein the method comprises the following steps:
attaching a test object, such as defined above, to the radiation head of the external radiation therapy treatment device, the test object being securely attached at a distance from the radiation source of the radiation head and as close as possible to the radiation head;
performing a pre-test by:
   irradiating the test object with a radiation beam emitted by the radiation head of the external radiation therapy treatment device during a sequence of rotation angles of the gantry of the external radiation therapy treatment device;
   acquiring a succession of images of the test object on the portal imager according to the sequence of rotation angles of the gantry of the external radiation therapy treatment device;
   measuring the size of the geometrical pattern formed by the radiopaque members of the test object and the position of the center of said pattern in all acquired images, using marks formed by the radiopaque members on the images after irradiating the test object;
   comparing the actual size of the geometrical pattern formed by the radiopaque members of the test object to the measured size of the geometrical pattern formed by the radiopaque members of the test object on each image, so as to obtain the distance between the radiation source and the portal imager (DSI), and thus the measure of the vertical motion artefacts of the portal imager with respect to the rotation angle of the gantry; and
   comparing the position of the center of the portal imager to the measured position of the center of the geometrical pattern formed by the radiopaque members of the test object on each image, so as to obtain the measure of the longitudinal and transverse motion artefacts of the portal imager with respect to the rotation angle of the gantry, the measure being corrected by the DSI variations calculated previously;
removing the test object from the radiation head;
acquiring the images corresponding to the performance test of the external radiation therapy treatment device; and
correcting the measures performed on these images, which measures characterize the performance of the external radiation therapy treatment device, with all motion artefacts of the portal imager using the measures performed during the pre-test.

This first method is advantageously used with a test object according to the first or third embodiment.

The images acquired during the pre-test according to the first method can be acquired in a static mode or in a cinema mode (images produced at regular intervals during the entire rotation of the gantry).

The invention also relates to a method without pre-test for measuring and correcting the measures performed in the images corresponding to the performance tests of an external radiation therapy treatment device to take into account the motion artefacts, with respect to the radiation source of the external radiation therapy treatment device, of a portal imager equipping the external radiation therapy treatment device when the latter is moving, the external radiation therapy treatment device further comprising a rotating gantry carrying at one end a radiation head comprising the radiation source and ending by a collimator allowing to delimit a radiation beam and, at the other end, a portal imager, wherein the method comprises the following steps:
attaching a test object, such as defined above, to the radiation head of the external radiation therapy treatment device, the test object being securely attached at a distance from the radiation source of the radiation head and as close as possible to the radiation head;
irradiating the test object, during the performance test of the external radiation therapy treatment device, by a radiation beam emitted by the radiation head of the external radiation therapy treatment device during a sequence of rotation angles of the gantry of the external radiation therapy treatment device, the collimator delimiting a transversally sliding slit formed by the two transversally opposite leaf banks to irradiate only one column of balls of both parallel rows of balls for a given rotation angle of the gantry;
acquiring the image of the test object on the portal imager during the entire sequence of rotation angles of the gantry, each column of the image being acquired for a given rotation angle of the gantry;
measuring, in the acquired image, the longitudinal distance separating, in each column of the image, the pair of balls corresponding to the associated rotation angle of the gantry and the position of the center of the pair of balls corresponding to the associated rotation angle of the gantry, using the marks created by the balls of both parallel rows of balls on the image after irradiating the test object;
in the acquired image, comparing the actual distance separating both rows of balls with the measured distance separating the pair of balls corresponding to the associated rotation angle of the gantry, so as to obtain the distance between the radiation source and the portal imager (DSI), and thus the measure of the vertical motion artefacts of the portal imager with respect to the rotation angle of the gantry;
in the acquired image, comparing the actual position of the center of the pair of balls corresponding to the associated rotation angle of the gantry with the measured position of the center of the pair of balls corresponding to the associated rotation angle of the gantry, so as to obtain the measure of the longitudinal and transverse motion artefacts of the portal imager with respect to the rotation angle of the gantry, the measure being corrected by the DSI variations calculated previously; and
in the acquired image, corresponding also to the performance test of the external radiation therapy treatment device, correcting the measures performed in the image, which measures characterize the performance of the external radiation therapy treatment device, with all motion artefacts of the portal imager using the measures performed previously.

This second embodiment is advantageously used with a test object according to the second or third embodiment. The acquired image according the second method is acquired in an integration mode (the produced image corresponding to the sum of radiation produced during the entire rotation movement of the gantry).

The method with pretest takes indirectly into account the motion artefacts, with respect to the radiation source of the external radiation therapy treatment device, of the portal imager, whereas the method without pretext takes these motions artefacts directly into account. The collimator comprises two transversally opposite banks, each bank comprising multiple leafs to delimit radiation beams with a complex profile. In the method without pretest, the collimator delimits, by its two transversally opposite leaf banks, a sliding slit which slides transversally during the rotation of the external radiation therapy treatment device.

### BRIEF DESCRIPTION OF THE SEVERAL VIEWS OF THE DRAWING

In order to better illustrate the subject-matter of the present invention, three preferred embodiments will be described thereafter, for illustrating and non-limiting purposes, with reference to the appended drawings, in which:

FIG. 1 shows a bottom view of a test object according to a first embodiment of the present invention;

FIG. 2 shows a bottom view of a test object according to a second embodiment of the present invention;

FIG. 3 shows a bottom view of a test object according to a third embodiment of the present invention;

FIG. 4 shows a perspective view of an external radiation therapy treatment device equipped with a test object according to the present invention;

FIG. 5 shows a schematic view of the superposition of the top view of the test object of the first embodiment of the present invention and of the associated image captured by the portal imager of the external radiation therapy treatment device at a given rotation angle of the gantry; and

FIG. 6 shows a schematic view of the superposition of the top view of the test object of the second embodiment of the present invention and of the associated image captured by the portal imager of the external radiation therapy treatment device at a given rotation angle of the gantry.

### DETAILED DESCRIPTION OF THE INVENTION

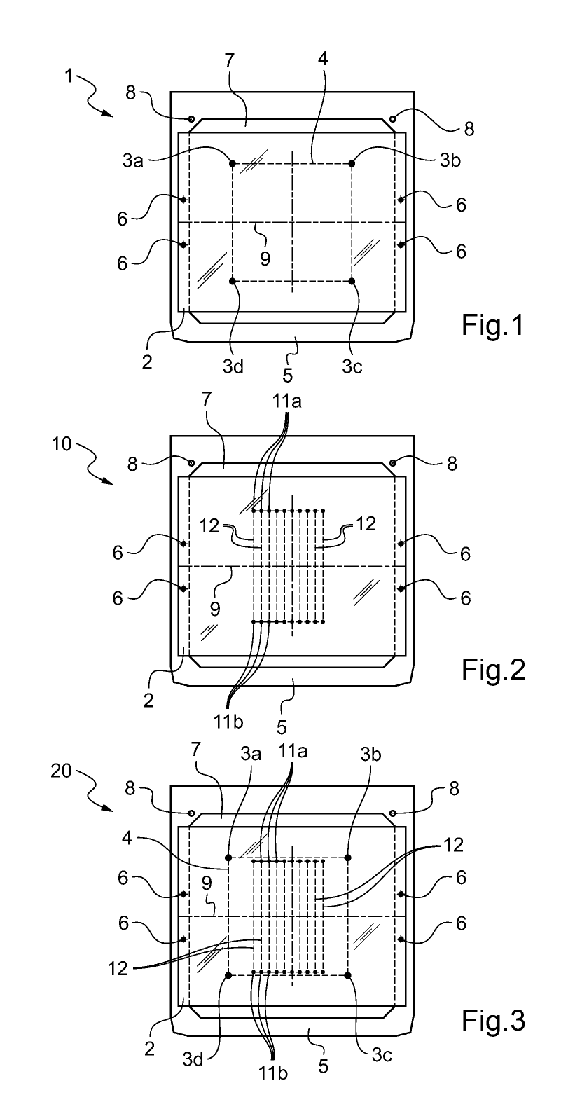

In reference to FIG. 1, a test object 1 according to a first embodiment of the invention is shown.

The test object 1 comprises a radiologically translucent plate 2 within the thickness of which four radiopaque members 3a, 3b, 3c, 3d are embedded.

The radiopaque members 3a, 3b, 3c, 3d are four metal balls arranged to form a square 4 (shown in dotted lines on FIG. 1 for illustrative purposes) on the radiologically translucent plate 2, the four radiopaque members 3a, 3b, 3c, 3d being at the same depth within the thickness of the radiologically translucent plate 2, two of the radiopaque members 3a, 3b being arranged at one of the edges of the radiologically translucent plate 2 and the two other radiopaque members 3c, 3d being arranged at the opposite edge of the radiologically translucent plate 2.

The radiologically translucent plate 2 is made of radiologically translucent plastic material, preferably polycarbonate.

It can be noted that the radiologically translucent plate 2 could also be made of carbon or any other sufficiently rigid material with an electronic density significantly lower than the electronic density of the radiopaque members 3a, 3b, 3c, 3d, especially an electronic density 2 times lower than the electronic density of the radiopaque members 3a, 3b, 3c, 3d, without departing from the scope of the present invention.

The thickness of the radiologically translucent plate 2 is constant and equal to 6 mm.

It can be noted that the thickness of the radiologically translucent plate 2 could be comprised between 3 and 10 mm, without departing from the scope of the present invention. The thickness should be sufficient for the radiologically translucent plate 2 to be sufficiently rigid not to be deformed during the rotation of the gantry on which it is attached, while being sufficiently radiologically translucent.

The radiopaque members 3a, 3b, 3c, 3d are made of tungsten carbide.

It can be noted that the radiopaque members 3a, 3b, 3c, 3d could also be made of any other metal or material with an electronic density significantly greater than that of the radiologically translucent plate 2, especially an electronic density twice higher than the electronic density of the radiologically translucent plate 2, without departing from the scope of the present invention.

For information purposes, the ratio of electronic density between the radiologically translucent plate 2 and the radiopaque members 3a, 3b, 3c, 3d is preferably between 2 and 20, most preferably between 10 and 20.

The diameter of the radiopaque members 3a, 3b, 3c, 3d is equal to 4 mm, the center of each radiopaque member 3a, 3b, 3c, 3d being at a depth, with respect to the upper face of the radiologically translucent plate 2 intended to face the radiation head of the external radiation therapy treatment device, equal to the radius of each radiopaque member 3a, 3b, 3c, 3d within the thickness of the radiologically translucent plate 2. Thus, the radiopaque members 3a, 3b, 3c, 3d substantially are flushed with said upper face of the radiologically translucent plate 2.

It can be noted that the diameter of the radiopaque members 3a, 3b, 3c, 3d could be between 1 and 10 mm, without departing from the scope of the present invention. Thus, the thickness of the radiologically translucent plate 2 would have to be adapted accordingly.

The test object 1 further comprises, in the embodiment shown, a hollow metal frame 5 comprising four edges on which the radiologically translucent plate 2 is attached, the radiologically translucent plate 2 being attached to the hollow metal frame 5 by laterally abutting the upper face of the plate 2 at two opposite edges of the hollow metal frame 5 via four screws 6 passing through corresponding holes formed on the radiologically translucent plate 2 and the hollow metal frame 5.

The hollow metal frame 5 comprises, in the center thereof, a hole 7 sized such that the radiopaque members 3a, 3b, 3c, 3d are within this hole 7 in a top view of the test object 1.

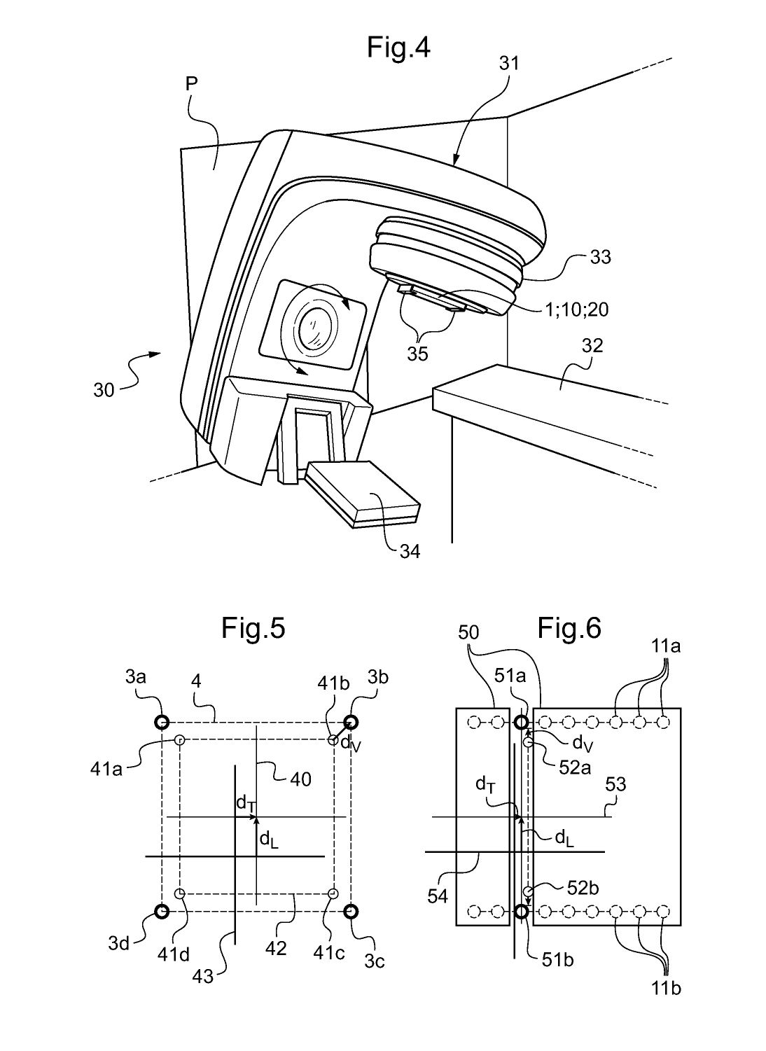

The dimensions in length and width of the radiologically translucent plate 2 are respectively inferior to those of the hollow metal frame 5, such that the radiologically translucent plate 2 does not disturb the insertion of the hollow metal frame 5 in a pair of slides (described in more detail in FIG. 4) intended to accommodate the frame 5.

Preferably, the radiologically translucent plate 2 has a width of 200 mm and a length of 254 mm and the hollow metal frame 5 has a width and a length of about 250 mm, preferably 264 mm, the hollow metal frame 5 being intended to be inserted in a pair of slides arranged at the output of the radiation head of a conventional external radiation therapy treatment device. The dimensions indicated above are not intended to be limitative, and the one skilled in the art will know how to adapt the dimensions of the plate to the dimensions of the external radiation therapy treatment device.

Preferably, the radiopaque members 3a, 3b, 3c, 3d are spaced apart from a distance of 134 mm on the edges of the square 4, the square 4 being centered on the radiologically translucent plate 2. Also, these dimensions are not intended tp be limitative, and the one skilled in the art will know how to adapt the dimensions such that the radiopaque members 3a, 3b, 3c, 3d are visible on the image (thus, in the field of the radiation beam) while not being on the useful center part formed on the portal imager.

The hollow metal frame 5 further comprises two holes 8 at two of its corners, so as to securely attach the frame 5 to the pair of slides of the conventional external radiation therapy treatment device via screws, after the test object 1 is completely inserted in the pair of slides.

Any other means for attaching the radiologically translucent plate 2 to the radiation head, other than the hollow metal frame 5, allowing to securely attach the radiologically translucent plate 2 to the radiation head, is contemplated within the scope of the present invention.

The radiologically translucent plate 2 further comprises a reticule 9 engraved on the upper face of the plate 2, the reticule 9 representing the center of the radiologically translucent plate 2.

It can be noted that the periphery of the radiologically translucent plate 2 is preferably machined and not sharp, in order to ensure handling of the plate 2.

In reference to FIG. 2, a test object 10 according to a second embodiment of the invention is shown.

The test object 10 comprises a radiologically translucent plate 2 within the thickness of which two parallel rows of radiopaque members 11a, 11b are embedded.

The radiopaque members are two parallel rows of metal balls 11a, 11b each comprising, in the example shown, ten metal balls (the number of balls being not limitative), the spacing between the ten balls of the first row of balls 11a being constant and equal to the spacing between the ten balls of the second row of balls 11b, the ten balls of the first row of balls 11a being aligned with respect to the ten balls of the second row of balls 11b such that the balls of the first and second rows of balls 11a, 11b are aligned by pairs in a parallel manner, in a direction perpendicular to the direction of both rows, (lines 12, representing each pair of balls of both rows of balls 11a, 11b are shown in FIG. 2 for illustrative purposes), the balls of both rows of balls 11a, 11b all being at the same depth within the thickness of the radiologically translucent plate 2, the first row of balls 11a being arranged at one of the edges of the radiologically translucent plate 2 and the second row of balls 11b being arranged at the opposite edge of the radiologically translucent plate 2.

It can be noted that each row of balls 11a, 11b could also comprise at least two balls, both rows of balls 11a, 11b having the same number of balls, without departing from the scope of the present invention.

The radiologically translucent plate 2 is made of radiologically translucent plastic material, preferably polycarbonate.

It can be noted that the radiologically translucent plate 2 could also be made of carbon or any other material with an electronic density significantly lower than the electronic density of the radiopaque members 11a, 11b, especially an electronic density 2 times lower than that of the radiopaque members 11a, 11b, without departing from the scope of the present invention. The thickness of the radiologically translucent plate 2 is constant and equal to 6 mm.

It can be noted that the thickness of the radiologically translucent plate 2 could be between 3 and 10 mm, without departing from the scope of the present invention.

The radiopaque members 11a, 11b are made of tungsten carbide.

It can be noted that the radiopaque members 11a, 11b could also be made of any other metal or material with an electronic density significantly greater than the electronic density of the radiologically translucent plate 2, especially an electronic density twice higher than the electronic density of the radiologically translucent plate 2, without departing from the scope of the present invention.

For information purposes, the electronic density ratio between the radiologically translucent plate 2 and the radiopaque members 11a, 11b is preferably between 2 and 20, most preferably between 2 and 10.

The diameter of the balls of both rows of balls 11a, 11b is equal to 2 mm, the center of each ball of both rows of balls 11a, 11b being at a depth, with respect to the upper face of the radiologically translucent plate 2, equal to the radius of each ball of both rows of balls 11a, 11b within the thickness of the radiologically translucent plate 2.

It can be noted that the diameter of the balls of both rows of balls 11a, 11b could be between 1 and 10 mm, without departing from the scope of the present invention. The thickness of the radiologically translucent plate 2 would have to be adapted accordingly.

The test object 10 further comprises a hollow metal frame 5 comprising four edges on which the radiologically translucent plate 2 is attached, the radiologically translucent plate 2 being attached on the hollow metal frame 5 by abutting laterally against the upper face of the plate 2 intended to face the radiation head on two opposite edges of the hollow metal frame 5 via four screws 6 passing through corresponding holes formed on the radiologically translucent plate 2 and the hollow metal frame 5.

A hole 7 is formed at the center of the hollow metal frame 5, the hole 7 being dimensioned such that the balls of both rows of balls 11a, 11b are within this hole 7 in a top view of the test object 10.

The dimensions in length and width of the radiologically translucent plate 2 are respectively inferior to those of the hollow metal frame 5, such that the radiologically translucent plate 2 does not disturb the insertion of the hollow metal frame 5 in a pair of slides intended to accommodate the frame 5.

Preferably, the radiologically translucent plate 2 has a width of 200 mm and a length of 254 mm and the hollow metal frame 5 has a width and a length of about 250 mm, preferably 264 mm, the hollow metal frame 5 being intended to be inserted in a pair of slides arranged at the output of the radiation head of a conventional external radiation therapy treatment device. The dimensions indicated above are obviously not limitative, and the one skilled in the art will know how to adapt the dimensions of the plate to the dimensions of the external radiation therapy treatment device.

Preferably, both rows of balls 11a, 11b are spaced apart from a distance of 123.6 mm, both rows of balls 11a, 11b being substantially centered on the radiologically translucent plate 2. Again, these dimensions are not limitative, and the one skilled in the art will know how to adapt the dimensions such that both rows of balls 11a, 11b are visible on the image (thus, within the field of the radiation beam) while not being on the useful center part of the image formed on the portal imager.

Preferably, the spacing between the balls of each row of balls 11a, 11b is of 8.6 mm, this spacing being intended for a use of the test object 20 on a conventional external radiation therapy treatment device comprising a collimator with two leaf banks allowing to form a longitudinal sliding slit (“picket fence”) with respect to the rotation angle of the gantry of the external radiation therapy treatment device, only one pair of balls being irradiated for a given rotation angle of the gantry.

The hollow metal frame 5 further comprises two holes 8 at two of its corners, so as to securely attach the frame 5 in the pair of slides of the conventional external radiation therapy treatment device via screws, after the test object 10 is completely inserted in the pair of slides.

The radiologically translucent plate 2 further comprises a reticule 9 engraved on the upper face of the plate 2, the reticule 9 representing the center of the radiologically translucent plate 2.

It can be noted that the periphery of the radiologically translucent plate 2 is preferably machined and not sharp, to ensure a secure handling of the plate 2.

In reference to FIG. 3, a test object 20 according to a third embodiment of the invention is shown.

It can be noted that this test object 20 is a combination of the test object 1 and of the test object 10.

The test object 20 comprises a radiologically translucent plate 2 within the thickness of which four radiopaque members 3a, 3b, 3c, 3d and two parallel rows of radiopaque members 11a, 11b are embedded.

The four radiopaque members 3a, 3b, 3c, 3d are four metal balls arranged to form a square 4 (shown in FIG. 3 for illustrative purposes) on the radiologically translucent plate 2, the four radiopaque members 3a, 3b, 3c, 3d all being at the same depth within the thickness of the radiologically translucent plate 2, two of the radiopaque members 3a, 3b being arranged on one of the edges of the radiologically translucent plate 2 and the two other radiopaque members 3c, 3d being arranged on the opposite edge of the radiologically translucent plate 2.

Both rows of radiopaque members 11a, 11b are two parallel rows of metal balls 11a, 11b similar to those of the second embodiment. Their detailed description is thus omitted here.

Both rows of balls 11a, 11b are arranged within the square 4 formed by the four radiopaque members 3a, 3b, 3c, 3d.

It can be noted that both rows of balls 11a, 11b could be partially or entirely out of the square 4 formed by the four radiopaque members 3a, 3b, 3c, 3d, without departing from the scope of the present invention.

The radiologically translucent plate 2 is made of radiologically translucent plastic material, preferably polycarbonate.

It can be noted that the radiologically translucent plate 2 could also be made of carbon or any other sufficiently rigid material with an electronic density significantly inferior to the electronic density of the radiopaque members 3a, 3b, 3c, 3d, and 11a, 11b, especially an electronic density twice lower than the electronic density of the radiopaque members 3a, 3b, 3c, 3d, and 11a, 11b, without departing from the scope of the present invention.

The thickness of the radiologically translucent plate 2 is constant and equal to 6 mm.

It can be noted that the thickness of the radiologically translucent plate 2 could be between 3 and 10 mm, without departing from the scope of the present invention, for the same reasons than the reasons indicated above in relation to the first and second embodiments.

The radiopaque members 3a, 3b, 3c, 3d and 11a, 11b are made of tungsten carbide.

It can be noted that the radiopaque members 3a, 3b, 3c, 3d and 11a, 11b could also be made of any other metal or material with an electronic density significantly greater than the electronic density of the radiologically translucent plate 2, namely an electronic density twice higher than the electronic density of the radiologically translucent plate 2, without departing from the scope of the present invention.

For information purposes, the electronic density ratio between the radiologically translucent plate 2 and the radiopaque members 3a, 3b, 3c, 3d and 11a, 11b is preferably between 2 and 20, most preferably between 2 and 10.

The diameter of the four radiopaque members 3a, 3b, 3c, 3d is equal to 4 mm, the center of each radiopaque member 3a, 3b, 3c, 3d being at a depth, with respect to the upper face of the radiologically translucent plate 2 facing the radiation head, equal to the radius of each radiopaque member 3a, 3b, 3c, 3d within the thickness of the radiologically translucent plate 2.

It can be noted that the diameter of the four radiopaque members 3a, 3b, 3c, 3d could be between 1 and 10 mm, without departing from the scope of the present invention, the thickness of the radiologically translucent plate 2 being adapted accordingly.

The diameter of the balls of both rows of balls 11a, 11b is equal to 2 mm, the center of each ball of both rows of balls 11a, 11b being at a depth, with respect to the upper face of the radiologically translucent plate 2 facing the radiation head, equal to the radius of each ball of both rows of balls 11a, 11b within the thickness of the radiologically translucent plate 2.

It can be noted that the diameter of the balls of both rows of balls 11a, 11b could be between 1 and 10 mm, without departing from the scope of the present invention.

The test object 20 further comprises a hollow metal frame 5 similar to that of the second embodiment, and its description is thus omitted here.

The dimensions in length and width of the radiologically translucent plate 2 are respectively inferior to the dimensions of the hollow metal frame 5, such that the radiologically translucent plate 2 does not disturb the insertion of the hollow metal frame 5 in a pair of slides intended to accommodate the frame 5.

Preferably, the radiologically translucent plate 2 has a width of 200 mm and a length of 254 mm and the hollow metal frame 5 has a width and a length of about 250 mm, preferably 264 mm, the hollow metal frame 5 being adapted to be inserted in a pair of slides arranged at the output of the radiation head of a conventional external radiation therapy treatment device. The dimensions indicated above are obviously not limitative, and the one skilled in the art will know how to adapt the dimensions of the plate to the dimensions of the external radiation therapy treatment device.

Preferably, the four radiopaque members 3a, 3b, 3c, 3d are spaced apart from a distance of 134 mm at the edges of the square 4, the square 4 being centered on the radiologically translucent plate 2.

Preferably, both rows of balls 11a, 11b are spaced apart from a distance of 123.6 mm, both rows of balls 11a, 11b being substantially centered on the radiologically translucent plate 2.

Preferably, the spacing between the balls of each row of balls 11a, 11b is of 8.6 mm, this spacing being adapted for a use of the test object 20 on a conventional external radiation therapy treatment device comprising a collimator with two leaf banks allowing to form a longitudinal sliding slit (“picket fence”) with respect to the rotation angle of the gantry of the external radiation therapy treatment device, only one pair of balls being irradiated for a given rotation angle of the gantry.

The hollow metal frame 5 further comprises two holes 8 at two of its corners, so as to securely attach the frame 5 in the pair of slides of the conventional external radiation therapy treatment device via screws, after the test object 20 is entirely inserted in the pair of slides.

The radiologically translucent plate 2 further comprises a reticule 9 engraved on the upper face of the plate 2, the reticule 9 representing the center of the radiologically translucent plate 2.

It can be noted that the periphery of the radiologically translucent plate 2 is machined and not sharp, to ensure a secure handling of the plate 2.

In reference to FIG. 4, a conventional external radiation therapy treatment device 30 is shown, equipped with a test object 1, 10, 20 according to the present invention, in an external radiation therapy treatment room.

This device 30 conventionally comprises a structure having at least one vertical wall P carrying a gantry 31, and a patient table or patient support 32.

The gantry 31, substantially U-shaped in a side view, is conventionally mounted on the vertical wall P, rotative around a horizontal axis. The gantry 31 carries at one end a radiation head 33 ending by a collimator and, at the other end, facing the collimator of the radiation head 33, a portal imager 34 able to detect a radiation emitted from the collimator of the radiation head 33, said portal imager 34 allowing to make pictures of the radiation emitted by the collimator and to perform the computer processing of the pictures.

The gantry 31 is free to rotate at 360° about a horizontal axis, said horizontal axis passing substantially through the center of the part in the vertical plane of the gantry 31, the zero rotation angle of the gantry 31 corresponding to the vertical position of the gantry 31.

The collimator internally comprises two leaf banks (not shown in FIG. 4) allowing to form a sliding slit (“picket fence”) by means of a simultaneous transverse translation of the benches, the transverse translation being coordinated with the rotation of the gantry 31. Thus, the collimator can emit only a part of the radiation from the radiation head 33 to the portal imager 34 via two leaf banks.

When the gantry 31 rotates, the portal imager 34 can also be moved in the three spatial dimensions by means of engines, gears and endless screws (not shown in FIG. 4).

The portal imager 34 is mainly used for capturing planar images of a patient positioned on the patient support 32. These images allow, before treating the patient, to control his/her position with respect to the radiation beam.

In this case, the portal imager 34 receives the radiation from the collimator which passed through the patient.

The portal imager 34 can also be used as an instrument for measuring the radiation emitted by the external radiation therapy treatment device 30 so as to control the performance of this device (quality control). In this case, the image is formed either with a beam only or with a beam which passed through a test object.

In both cases, any analysis performed by the portal imager 34 on the images is based first on distance measures within the images.

The external radiation therapy treatment device 30 further comprises a pair of slides 35 arranged at the output of the collimator of the radiation head 33, and a test object 1, 10, 20 can be inserted in said pair of slides 35 such that the test object 1, 10, 20 is arranged as close as possible to the radiation head 33 of the external radiation therapy treatment device 30, the test object 1, 10, 20 being securely attached once inserted in the pair of slides 35 via screws, such that the test object 1, 10, 20 does not move with respect to the radiation source when the gantry 31 rotates.

The portal imager 34 can be used according to three operating modes: a static mode in which the image is acquired by the portal imager 34 when the external radiation therapy treatment device 30 is stationary, a cinema mode in which images are acquired by the portal imager 34 at regular intervals during the entire rotation of the gantry 31 of the external radiation therapy treatment device 30, and an integration mode in which the image acquired by the portal imager 34 corresponds to the sum of the radiation during the entire rotation of the gantry 31 of the external radiation therapy treatment device 30.

In the cinema and integration modes, the images acquired by the portal imager 34 have artefacts induced by the motion artefacts (parasitic movement) of the portal imager 34 when the portal imager 34 is in movement with the rotation of the gantry 31 of the external radiation therapy treatment device 30.

To measure the motion artefacts of the portal imager 34 with respect to the radiation source contained in the head of the external radiation therapy treatment device so as to compensate them, a test object 1, 10, 20 is inserted in the pair of slides 35 arranged at the output of the collimator, the test object 1, 10, 20 comprising radiopaque members for measuring the vertical, longitudinal and transverse motion artefacts of the portal imager 34 with respect to the collimator during performance tests of the multileaf collimator used in a dynamic mode for both delimiting the radiation beam and modulating its intensity during the treatment.

In reference to FIG. 5, the superposition of the top view of the test object 1 of the first embodiment of the present invention and of the associated image captured by the portal imager 34 of the external radiation therapy treatment device 30 at a given rotation angle of the gantry 31 is shown.

The test object 1 of the first (or third) embodiment of the present invention can be used according to a method with pre-test for measuring and correcting measures performed in the images corresponding to the performance tests of an external radiation therapy treatment device 30 to take into account the motion artefacts of a portal imager 34 equipping said external radiation therapy treatment device 30 when the latter is moving, the external radiation therapy treatment device 30 further comprising a rotating gantry 31 carrying at one end a radiation head 33 and, at the other end, a portal imager 34, comprising the following steps:
attaching a test object 1, 20, according to the first or third embodiment, to the radiation head 33 of the external radiation therapy treatment device 30, the test object 1, 20 being securely attached at a distance from the radiation source of the radiation head 33 and as close as possible to the radiation source;
performing a pre-test by:
   irradiating the test object 1, 20 with a radiation beam emitted from the radiation head 33 of the external radiation therapy treatment device 30 during a sequence of rotation angles of the gantry 31 of the external radiation therapy treatment device 30;
   acquiring a succession of images of the test object 1, 20 on the portal imager 34 according to the sequence of rotation angles of the gantry 31 of the external radiation therapy treatment device 30;
   measuring the size of the geometrical pattern formed by the balls 3a, 3b, 3c, 3d of the test object 1, 20 and the position of the center of said pattern in all acquired images, using marks created by the balls 3a, 3b, 3c, 3d in the images after irradiating the test object 1, 20;
   comparing the actual size of the geometrical pattern formed by the balls 3a, 3b, 3c, 3d of the test object 1, 20 with the measured size of the geometrical pattern formed by the balls 3a, 3b, 3c, 3d of the test object 1, 20 on each image, so as to obtain the distance between the radiation source and the portal imager DSI, and thus the measure of the vertical motion artefacts of the portal imager 34 with respect to the rotation angle of the gantry 31; and
   comparing the position of the center of the portal imager 34 with the measured position of the center of the geometrical pattern formed by the balls 3a, 3b, 3c, 3d of the test object 1, 20 on each image, so as to obtain the measure of the longitudinal and transverse motion artefacts of the portal imager 34 with respect to the rotation angle of the gantry 31, the measure being corrected with the DSI variations calculated previously;
removing the test object 1, 20 from the radiation head 33;
acquiring the images corresponding to the performance test of the external radiation therapy treatment device; and
correcting the measures performed in these images, which measures characterize the performance of the external radiation therapy treatment device, with all motion artefacts of the portal imager 34 using the measures performed during the pre-test.

It can be noted that the method with pre-test can be used only when the motion artefacts of the portal imager 34 can be reproduced between the pre-test and the test.

FIG. 5 shows the superposition of the top view of the test object 1 of the first embodiment of the present invention and of the associated image captured by the portal imager 34 of the external radiation therapy treatment device at a given rotation angle of the gantry 31 during the method with pre-test.

As mentioned above, the radiopaque members 3a, 3b, 3c, 3d of the test object 1 form a square 4, the center of the square being represented in an illustrative manner in FIG. 5 by a cross 40. After irradiating the test object 1 by the radiation head 33, the portal imager 34 acquires images 41a, 41b, 41c, 41d of the radiopaque members 3a, 3b, 3c, 3d, the images 41a, 41b, 41c, 41d of the radiopaque members 3a, 3b, 3c, 3d forming a square 42, the size of which is different from the size of the square 4 (in FIG. 5, the images 41a, 41b, 41c, 41d of the radiopaque members 3a, 3b, 3c, 3d and the square 42 being moved so as to be centered on the center of the cross 40 for understanding purposes), the size difference between the square 4 and the square 42 allowing to measure the distance between the radiation source and the portal imager (called DSI), and thus the measure of the vertical motion artefacts d_{V} of the portal imager 34 with respect to the rotation angle of the gantry 31.

Then, the center of the square formed by the images 41a, 41b, 41c, 41d of the radiopaque members 3a, 3b, 3c, 3d is represented in FIG. 5 by a cross 43, the difference of position between the center of the portal imager 34 and the center of the cross 43, after correcting the DSI variation, allowing to measure the longitudinal motion artefacts d_{L} and the transverse motion artefacts d_{T} of the portal imager 34 with respect to the rotation angle of the gantry 31.

In reference to FIG. 6, the superposition of the top view of the test object 10 of the second embodiment of the present invention and of the associated image captured by the portal imager 34 of the external radiation therapy treatment device 30 at a given rotation angle of the gantry 31 is shown.

The test object 10 of the second (or third) embodiment of the present invention can be used according to a method without pre-test for measuring and correcting the measures performed in the images corresponding to the performance tests of an external radiation therapy treatment device 30 to take into account the motion artefacts of a portal imager 34 equipping said external radiation therapy treatment device 30 when the latter is moving, the external radiation therapy treatment device 30 further comprising a rotating gantry 31 carrying at one end a radiation head 33 ending by a collimator allowing to delimit a radiation beam and, at the other end, a portal imager 34, wherein the method comprises the following steps:
attaching a test object 10, 20 on the radiation head 33 of the external radiation therapy treatment device 30, the test object 10, 20 being securely attached at a distance from the radiation source of the radiation head 33 and as close as possible to the radiation source;
irradiating the test object 10, 20, during the performance test of the external radiation therapy treatment device, with a radiation beam emitted from the radiation head of the external radiation therapy treatment device 30 during a sequence of rotation angles of the gantry 31 of the external radiation therapy treatment device 30, the collimator delimiting a transversally sliding slit formed by two leaf banks to irradiate only one column of balls of both parallel rows of balls 11a, 11b for a given rotation angle of the gantry 31;
acquiring the image of the test object 10, 20 on the portal imager 34 used in an integration mode during the entire sequence of rotation angles of the gantry 31, each column of the image being acquired for a given rotation angle of the gantry 31;
measuring, in the acquired image, the longitudinal distance separating, in each column of the image, the pair of balls 51a, 51b corresponding to the associated rotation angle of the gantry 31 and the position of the center of the pair of balls 51a, 51b corresponding to the associated rotation angle of the gantry 31, using the marks created by the balls of both parallel rows of balls 11a, 11b on the image after irradiating the test object 10, 20;
in the acquired image, comparing the actual distance separating both rows of balls 11a, 11b to the measured distance separating the pair of balls 51a, 51b corresponding to the associated rotation angle of the gantry 31, so as to obtain the distance between the radiation source and the portal imager (DSI), and thus the measure of the vertical motion artefacts of the portal imager 34 with respect to the rotation angle of the gantry 31;
in the acquired image, comparing the actual position of the center of the pair of balls 51a, 51b corresponding to the associated rotation angle of the gantry to the measured position of the center of the pair of balls 51a, 51b corresponding to the associated rotation angle of the gantry 31, so as to obtain the measure of the longitudinal and transverse motion artefacts of the portal imager 34 with respect to the rotation angle of the gantry 31, the measure being corrected with the DSI variations calculated previously; and
in the acquired image, corresponding also to the performance test of the external radiation therapy treatment device, correcting the measures performed in the image and which characterize the performance of the external radiation therapy treatment device, with all motion artefacts of the portal imager 34 using the measures performed previously.

It can be noted that this method without pre-test is valid even when the motion artefacts of the portal imager 34 cannot be reproduced.

FIG. 6 shows the superposition of the top view of the test object 10 of the second embodiment of the present invention and of the associated image captured by the portal imager 34 of the external radiation therapy treatment device at a given rotation angle of the gantry 31, during the method without pre-test.

As mentioned above, the radiopaque members of the test object 10 are two parallel rows of balls 11a, 11b. For each angle of the gantry 31, both leaf banks 50 of the collimator are positioned to irradiate only one pair of balls 51a, 51b of both rows of balls 11a, 11b. After irradiating the test object 10 by the radiation head 33, the portal imager 34 acquires images 52a, 52b of the pair of balls 51a, 52b, the longitudinal distance between the images 52a, 52b of the pair of balls 51a, 51b differing from the distance between the balls of the pair of balls 51a, 51b (in FIG. 6, the images 52a, 52b of the pair of balls 51a, 51b being moved between the pair of balls 51a, 51b for a better understanding), the difference of longitudinal distance between the images 52a, 52b of the pair of balls 51a, 51b and the balls of the pair of balls 51a, 51b allowing to measure the distance between the radiation source and the portal imager (DSI), and thus the measure of the vertical motion artefacts d_{V} of the portal imager 34 with respect to the rotation angle of the gantry 31.

Then, the centers of the pair of balls 51a, 51b and of the images 52a, 52b of the pair of balls 51a, 51b are marked in FIG. 6 by a cross 53 and a cross 54, respectively, the difference of position between the center of the cross 53 and the center of the cross 54, after correcting the DSI variation, allowing to measure the longitudinal motion artefacts d_{L} and the transverse motion artefacts d_{T} of the portal imager 34 with respect to the rotation angle of the gantry 31.

It can be noted that the test object 20 of the third embodiment of the present invention can be used according to the method with pre-test by using the four radiopaque members 3a, 3b, 3c, 3d or to the method without pre-test by using both rows of balls 11a, 11b.

The correction of the measures performed in the images corresponding to the performance tests to take into account the motion artefacts of the portal imager 34, when acquiring the image, can be performed according to two methods: a method for integrally correcting the image of the three types of motion artefacts (transverse, longitudinal and vertical) of the portal imager 34, and a method for correcting only the DSI parasitic variations due to the vertical motion artefacts of the distance or intensity measures performed in the non-corrected images.

The method of integral correction is possible only on images transversally formed as the gantry 31 rotates by means of the transverse progression of a sliding slit delimited by both leaf banks 50 of the collimator. In this method, all pixels of a column of this image type being acquired for a same rotation angle of the gantry 31, the correction of the image consists in transversally and longitudinally moving the pixel column according to the angle of the gantry 31, the measured movements being previously scaled by applying the “nominal DSI/measured DSI” factor. Furthermore, the intensity of each pixel of the pixel column is corrected by the law of the inverse of the square of the distance from the radiation source, namely the inverse of the square of DSI corresponding to the rotation angle of the gantry 31 associated with the column of the image to which the pixel belongs, this intensity correction being required for taking into account the fact that, if the portal imager 34 moves away, the amount of X rays arriving on the portal imager 34 is reduced according to the inverse of the square of the distance with respect to the radiation source. Finally, the resulting image corresponds to a resegmentation on the initial pixel frame of the moved pixel columns.

The method for correcting only the DSI variations due to the motion artefacts is not a correction of images, but a correction of measures of distance or signal intensity performed in the non-corrected images. This method of partial correction is applicable and required: i) when the analysis of an image acquired when the gantry 31 rotates is based only on comparisons of signal intensities between different image regions acquired at different rotation angles of the gantry 31, ii) when the analysis consists in comparing signal levels between different images acquired at different fixed rotation angles of the gantry 31, and iii) when the analysis consists in comparing distances with respect to the center of the beam performed on different images acquired when the gantry 31 is stationary, but for different rotation angles of the gantry 31. In cases i) and ii), the intensity of the measured signals is corrected by bringing the signals back to the nominal DSI using the law of the inverse of the square of the distance from the radiation source, and in the case iii), all distances are brought back to the nominal DSI by applying the following correction on the measured distances: (distance corrected to the nominal DSI)=(distance measured from the DSI)*(nominal DSI/DSI).

1. A test object for an external radiation therapy treatment device, said external radiation therapy treatment device comprising a rotating gantry carrying at one end a radiation head comprising a radiation source and, at the other end, a portal imager, the test object being intended to correct motion artefacts of the portal imager with respect to the radiation source during a movement of the external radiation therapy treatment device, wherein the test object comprises a radiologically translucent plate within the thickness of which at least one radiopaque member is embedded, the at least one radiopaque member forming a geometrical pattern having at least two points and the center of the geometrical pattern corresponding to the center of the radiologically translucent plate, fastening means allowing to attach the test object on the radiation head, and wherein the radiologically translucent plate carries two parallel rows of balls, each row of balls comprising at least two balls, both rows of balls comprising the same number of balls spaced with the same spacing, such that the line connecting the balls facing each other in both rows is perpendicular to the direction of the rows of balls.

2. The test object according to claim 1, wherein the test object further comprises at least four radiopaque balls forming one of a square or a rectangle, whose contour surrounds both rows of balls, the center of the square or rectangle formed by the four radiopaque balls corresponding to the barycenter of both rows of balls.

3. The test object according to claim 1, wherein the radiologically translucent plate is made of radiologically translucent plastic material, preferably polycarbonate.

4. The test object according to claim 1, wherein the thickness of the radiologically translucent plate is constant and comprised between 3 and 10 mm, preferably 6 mm.

5. The test object according to claim 1, wherein the radiopaque members are made of tungsten carbide.

6. The test object according to claim 1, wherein each radiopaque ball is embedded in the radiologically translucent plate such that the center of each radiopaque ball is at a distance from the face of the radiologically translucent plate equal to the radius of the radiopaque ball.

7. The test object according claim 1, wherein a reticule is engraved on the face of the radiologically translucent plate intended to face the radiation head, the reticule representing the center of the radiologically translucent plate.

8. A method with pre-test for measuring and correcting the measures performed in the images corresponding to the performance tests of an external radiation therapy treatment device to take into account the motion artefacts of a portal imager equipping the external radiation therapy treatment device with respect to the radiation source of the external radiation therapy treatment device when the external radiation therapy treatment device is moving, the external radiation therapy treatment device further comprising a rotating gantry carrying at one end a radiation head comprising the radiation source and, at the other end, a portal imager, wherein the method comprises the following steps:
attaching a test object, such as defined in claim 1, to the radiation head of the external radiation therapy treatment device, the test object being securely attached at a distance from the radiation source of the radiation head and as close as possible to the radiation source;
performing a pre-test by:
irradiating the test object with a radiation beam emitted from the radiation head of the external radiation therapy treatment device during a sequence of rotation angles of the gantry of the external radiation therapy treatment device;
acquiring a succession of images of the test object on the portal imager according to the sequence of rotation angles of the gantry of the external radiation therapy treatment device;
measuring the size of the geometrical pattern formed by the balls of the test object and the position of the center of the geometrical pattern in all acquired images, using marks created by the balls on the images after irradiating the test object;
comparing the actual size of the geometrical pattern formed by the balls of the test object to the measured size of the geometrical pattern formed by the balls of the test object on each image, so as to obtain the distance between the radiation source and the portal imager (DSI), and thus the measure of the vertical motion artefacts of the portal imager with respect to the rotation angle of the gantry; and
comparing the position of the center of the portal imager to the measured position of the center of the geometrical pattern formed by the balls of the test object on each image, so as to obtain the measure of the longitudinal and transverse motion artefacts of the portal imager with respect to the rotation angle of the gantry, the measure being corrected with the DSI variations calculated at prior step;
removing the test object from the radiation head;
acquiring the images corresponding to the performance test of the external radiation therapy treatment device; and
correcting the measures performed in these images and which characterize the performance of the external radiation therapy treatment device with all motion artefacts of the portal imager using the measures performed during the pre-test.

9. A method without pre-test for measuring and correcting the measures performed in the images corresponding to the performance tests of an external radiation therapy treatment device to take into account the motion artefacts of a portal imager equipping the external radiation therapy treatment device with respect to the radiation source of the external radiation therapy treatment device when the external radiation therapy treatment device is moving, the external radiation therapy treatment device further comprising a rotating gantry carrying at one end a radiation head comprising the radiation source ending by a collimator comprising two transversally opposite leaf banks, each leaf bank comprising at least two leafs, the leaf banks allowing to delimit a radiation beam with a complex profile and, at the other end, a portal imager, wherein the method comprises the following steps:
attaching a test object, such as defined in claim 1, to the radiation head of the external radiation therapy treatment device, the test object being securely attached at a distance from the radiation source of the radiation head and as close as possible to the radiation source;
irradiating the test object, during the performance test of the external radiation therapy treatment device, with a radiation beam emitted from the radiation head of the external radiation therapy treatment device during a sequence of rotation angles of the gantry of the external radiation therapy treatment device, the collimator delimiting a transversally sliding slit formed by the two transversally opposite leaf banks to irradiate only one column of balls of both parallel rows of balls for a given rotation angle of the gantry;
acquiring the image of the test object on the portal imager during the entire sequence of rotation angles of the gantry, each column of the image being acquired for a given rotation angle of the gantry;
measuring, in the acquired image, the longitudinal distance separating, in each column of the image, the pair of balls corresponding to the associated rotation angle of the gantry and the position of the center of the pair of balls corresponding to the associated rotation angle of the gantry, using the marks created by the balls of both parallel rows of balls on the image after irradiating the test object;
in the acquired image, comparing the actual distance separating both rows of balls to the measured distance separating the pair of balls corresponding to the associated rotation angle of the gantry, so as to obtain the distance between the radiation source and the portal imager (DSI), and thus the measure of the vertical motion artefacts of the portal imager with respect to the rotation angle of the gantry;
in the acquired image, comparing the actual position of the center of the pair of balls corresponding to the associated rotation angle of the gantry to the measured position of the center of the pair of balls corresponding to the associated rotation angle of the gantry, so as to obtain the measure of the longitudinal and transverse motion artefacts of the portal imager with respect to the rotation angle of the gantry, the measure being corrected with the DSI variations calculated at the prior step; and
in the acquired image, corresponding also to the performance test of the external radiation therapy treatment device, correcting the measures performed in the image and which characterize the performance of the external radiation therapy treatment device, with all motion artefacts of the portal imager using the measures performed at prior step.
